(19) United States
(12) Patent Application Publication
(10) Pub. No.: US 2007/0191261 A1
(43) Pub. Date: Aug. 16, 2007

(54) Hairscents with anti-dandruff formula

(76) Inventor: Jaele Davis, Westchester, CA (US)

Correspondence Address: FULWIDER PATTON LLP, HOWARD HUGHES CENTER, 6060 CENTER DRIVE, TENTH FLOOR, LOS ANGELES, CA, 90045, US

(21) Appl. No.: 11/490,866

(22) Filed: Jul. 21, 2006

Related U.S. Application Data
(63) Continuation-in-part of application No. 11/414,809, filed on May 1, 2006.
(60) Provisional application No. 60/703,251, filed on Jul. 29, 2005.
(60) Provisional application No. 60/676,982, filed on May 2, 2005.

(51) Int. Cl. A61K 8/64

(52) U.S. Cl. 514/2; 424/70.14; 424/47

(57) ABSTRACT

A product for eliminating odors that can accumulate in a person's hair and to address itching and flaking associated with dandruff.

### CROSS-REFERENCES TO RELATED APPLICATIONS

This application is based on Provisional Application Ser. No. 60/703,251, filed Jul. 29, 2005, and is a continuation-in-part of U.S. application Ser. No. 11/414,809, filed May 1, 2006, which is based on Provisional Application Ser. No. 60/676,982, filed May 2, 2005.

### BACKGROUND OF THE INVENTION

Environmental and personal factors can add undesirable odors to otherwise clean hair. Often, individuals go to a restaurant or other public place and when they leave, their hair smells like cigarette smoke, cooking oils and food, pollution or other environmental elements. Certain hairstyles, such as braids, braided or bonded weaves, beaded designs and dreadlocks, prohibit daily washing due to their intricate nature, but it is also great for hairstyles that require blow drying, flat ironing and wavy styles. It is great for people who work out in the gym regularly and want to freshen up their hair as they continue on with their errands and don't have time to wash their hair or go to the hair salon. Currently, no products are offered that provide a fresh scent and eliminate common odors that can occur between washings.

Additionally, environmental and personal factors can add undesirable itching and flaking, commonly known as dandruff, to hair. Often, individuals accumulate dandruff as a result of personal health issues, while others are faced with a dry scalp. Certain hairstyles, such as braids, braided weaves, bonded weaves, beaded designs and dreadlocks, prohibit daily washing due to their intricate nature, but it is also great for hairstyles that require blow drying, flat ironing and wavy styles. It is great for people who want to combat dandruff and have fresh smelling hair in between washes and hair salon visits. What is needed is a formula that eliminates itching and flaking of dandruff, while providing a fresh scent and disposing of common odors like cigarette smoke, cooking oils, food, pollution or other environmental elements that can occur between washings.

The present invention satisfies these and other needs.

### SUMMARY OF THE INVENTION

In one aspect, HairScents is designed to eliminate odors that can accumulate on the hair in between washes. This unique invention contains ingredients that add protein and vitamins to the hair while eliminating the odors that become trapped in the hair follicles. This produce is offered in a variety of scents sure to please all consumers.

In another aspect, HairScents Anti-Dandruff Formula is designed to eliminate the itching and flaking associated with dandruff while providing a variety of fresh scents to combat odors that can accumulate on the hair in between washes. This unique invention contains ingredients that add protein and vitamins to the hair while eliminating dandruff and the odors that become trapped in the hair follicles. This product is offered in a variety of scents sure to please all consumers.

Other features and advantage of the present invention will become apparent from the following detailed description, taken in conjunction with the accompanying drawings, which illustrate, by way of example, the principles of the invention.

### BRIEF DESCRIPTION OF THE DRAWINGS

FIG. 1 is a schematic representation, depicting the present invention directed towards hair scents.

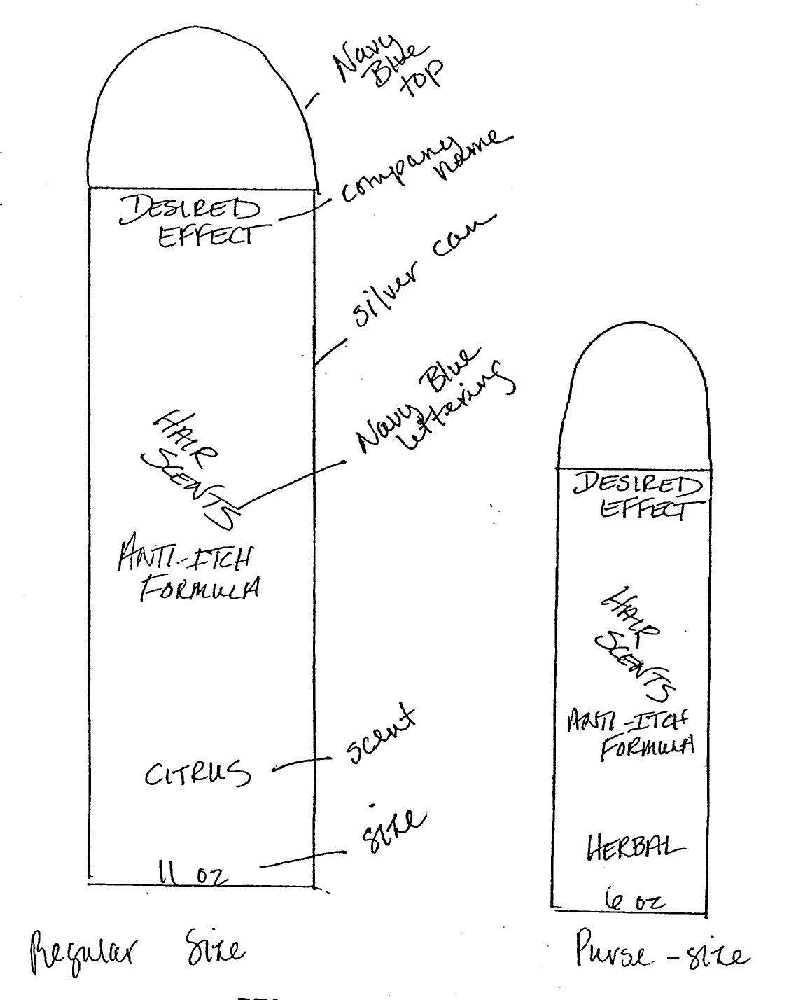

### DETAILED DESCRIPTION OF THE PREFERRED EMBODIMENTS

HairScents is designed to refresh the aroma of hair between washes. This product is a spray-on formula that can be offered in a variety of sizes. The typical sizes will include a container that is approximately 10″ tall an a smaller purse size can/bottle that is expected to be between 5-7″ in height. The spray contains ingredients that add protein and vitamins to the hair with no heavy product build-up. Scents are added to the formula to result in the hair smelling clean and fresh. The scents can include, but are not limited to: baby powder, citrus, fresh flowers and herbal fragrances. The container can be offered with a distinctive design that has an aluminum or silver appearance with navy blue and black lettering on the can. A blue cap or top may also distinguish this product from other hair care products on the market. To use: simply spray throughout dry hair and style as usual.

HairScents Anti-Dandruff Formula is designed to eliminate dandruff and the itching and flaking associated with dandruff, while refreshing the aroma of hair between washes. This product is a spray-on aerosol formula that can be offered in a variety of sizes. The typical sizes will include a can/bottle that is approximately 10″ tall and a smaller purse size can/bottle that is expected to be between 5-7″ in height. The spray contains ingredients that eliminate dandruff, while adding protein and vitamins to the hair with no heavy build-up. Scents are added to the formula to result in the hair smelling clean and fresh. The scents can include, but are not limited to, citrus, herbal, fresh flowers and baby powder fragrances. The container can be offered with a distinctive design that has an aluminum or silver appearance with navy blue and black lettering on the can/bottle. A navy blue cap or top may also distinguish this product from other hair care products on the market. To use: simply spray throughout dry hair and style as usual.

Thus, it will be apparent from the foregoing that, while particular forms of the invention have been illustrated and described, various modifications can be made without parting from the spirit and scope of the invention.

We claim:

1. A hair product, comprising:
a container, the container including means for spraying material stored in the container; and
a spray-on formula stored in the container, the formula including a protein and a plurality of vitamins wherein the formula eliminates odors trapped in hair follicles.

2. The hair product of claim 1, further comprising components which facilitate the elimination of dandruff and itching and flaking associated therewith.
